(19) United States
(12) Patent Application Publication
(10) Pub. No.: US 2025/0072796 A1
(43) Pub. Date: Mar. 6, 2025

(54) BIOLOGICAL INFORMATION ACQUISITION DEVICE, BIOLOGICAL INFORMATION ACQUISITION METHOD, AND RECORDING MEDIUM

(71) Applicant: NEC Corporation, Minato-ku, Tokyo (JP)

(72) Inventors: Yusuke AKAMATSU, Tokyo (JP); Yoshifumi ONISHI, Tokyo (JP)

(73) Assignee: NEC Corporation, Minato-ku, Tokyo (JP)

(21) Appl. No.: 18/289,608

(22) PCT Filed: Oct. 18, 2022
(86) PCT No.: PCT/JP2022/038694

§ 371 Date: Nov. 6, 2023

(51) Int. Cl. A61B 5/1455; A61B 5/00

(52) U.S. Cl. CPC A61B 5/14552; A61B 5/7203; A61B 5/7267; A61B 5/7278; A61B 5/7485; A61B 2576/00

(57) ABSTRACT

In the biological information acquisition device, the spatiotemporal information generation means generates spatiotemporal information by accumulating information relating to a region of interest extracted from a plurality of time-series images included in a biological video obtained by shooting a skin of a subject for a predetermined period. The extraction means extracts AC components and DC components from the spatiotemporal information. The estimation means estimates blood oxygen saturation of the subject in the predetermined period, based on the AC components and the DC components. This device can be used to support user's decision making and the like.

### TECHNICAL FIELD

The present disclosure relates to a technique for acquiring biological information based on a video obtained by shooting a human body.

### BACKGROUND ART

Percutaneous arterial oxygen saturation (hereafter also referred to as SpO2) is conventionally known as an indicator of how much oxygen is contained in blood.

Further, for example, Non-Patent Document 1 discloses a technique for estimating SpO2 of the human body by acquiring time series signals of three channels of red, green, and blue from a video shooting a human body and inputting the acquired time series signals into a deep learning model.

### PRECEDING TECHNICAL REFERENCES

### Non-Patent Document

Non-Patent Document 1:
Joshua Mathew, et. al, “Remote Blood Oxygen Estimation From Videos Using Neural Networks”, [online], May 5, 2022, arXiv, [Searched on Sep. 20, 2022], Internet <URL: https://arxiv.org/pdf/2107.05087.pdf>

### SUMMARY

### Problem to be Solved

Here, according to the technique disclosed in Non-Patent Document 1, SpO2 is estimated without explicitly using AC and DC components included in the time series signals of the three channels. AC and DC components are key elements in SpO2 measurement principles. Therefore, according to the technique disclosed in Non-Patent Document 1, there is a possibility that a limit exists in the accuracy of estimating the blood oxygen saturation.

It is an object of the present disclosure to provide a biological information acquisition device capable of improving estimation accuracy of blood oxygen saturation.

### Means for Solving the Problem

According to an example aspect of the present invention, there is provided a biological information acquisition device comprising:
a spatiotemporal information generation means configured to generate spatiotemporal information by accumulating information relating to a region of interest extracted from a plurality of time-series images included in a biological video, the biological video being obtained by shooting a skin of a subject for a predetermined period;
an extraction means configured to extract AC components and DC components from the spatiotemporal information; and
an estimation means configured to estimate blood oxygen saturation of the subject in the predetermined period, based on the AC components and the DC components.

According to another example aspect of the present invention, there is provided a biological information acquisition method comprising:
generating spatiotemporal information by accumulating information relating to a region of interest extracted from a plurality of time-series images included in a biological video, the biological video being obtained by shooting a skin of a subject for a predetermined period;
extracting AC components and DC components from the spatiotemporal information; and
estimating blood oxygen saturation of the subject in the predetermined period, based on the AC components and the DC components.

According to still another example aspect of the present invention, there is provided a recording medium storing a program, the program causing a computer to execute processing comprising:
generating spatiotemporal information by accumulating information relating to a region of interest extracted from a plurality of time-series images included in a biological video, the biological video being obtained by shooting a skin of a subject for a predetermined period;
extracting AC components and DC components from the spatiotemporal information; and
estimating blood oxygen saturation of the subject in the predetermined period, based on the AC components and the DC components.

### Effect

According to the present disclosure, it is possible to improve the accuracy of estimating the blood oxygen saturation.

### BRIEF DESCRIPTION OF THE DRAWINGS

FIG. 1 is a diagram showing a schematic configuration of a biological information acquisition system including a biological information acquisition device according to a first example embodiment.

FIG. 2 is a block diagram showing a hardware configuration of the biological information acquisition device according to the first example embodiment.

FIG. 3 is a block diagram showing a functional configuration of a biological information acquisition device according to the first example embodiment.

FIG. 4 is a block diagram for explaining a specific configuration of the biological information acquisition device according to the first example embodiment.

FIG. 5 shows a specific example of a deep learning model used for extracting AC components of and a DC components by the biological information acquisition device according to the first example embodiment.

FIG. 6 is a flowchart illustrating an example of processing performed in the biological information acquisition device according to the first example embodiment.

FIG. 7 is a block diagram showing a functional configuration of a biological information acquisition device according to a second example embodiment.

FIG. 8 is a flowchart for explaining processing performed in the biological information acquisition device according to the second example embodiment.

### EXAMPLE EMBODIMENTS

Preferred example embodiments of the present invention will be described with reference to the accompanying drawings.

### <Basic Principle>

As a principle for estimating SpO2, Ratio-of-Rations (RoR) method is known. RoR method calculates SpO2 by calculating a ratio of the AC components and the DC components of the red and blue channels in a video taken at a predetermined position of the human body. A technique for calculating SpO2 using RoR method is disclosed in L. Tarassenko, et. al, “Non-contact video-based vital sign monitoring using ambient light and auto-regressive models,” Physiological measurement, vol. 35, no. 5, pp. 807, 2014, for example. A technique for calculating SpO2 using RoR method is also disclosed in Hamidur Rahman, et. al, “Non-Contact Physiological Parameters Extraction Using Facial Video Considering Illumination, Motion, Movement and Vibration,” IEEE Transactions on Biomedical Engineering, vol. 67, no. 1, pp. 88-98, 2019, for example. Here, it is known that the AC component represents the pulsating component and the DC component represents the non-pulsating component. In the following example embodiments, in consideration of RoR method, the estimation of SpO2 is performed by extracting AC and DC components of the red, green, and blue channels in the video obtained by shooting a predetermined position of a human body.

### First Example Embodiment

### [System Configuration]

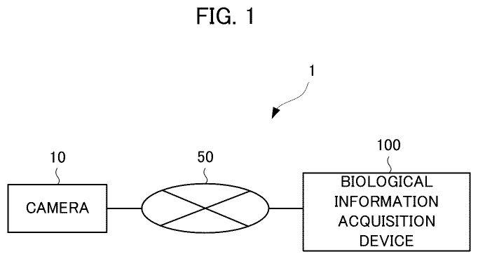

FIG. 1 is a diagram showing a schematic configuration of a biological information acquisition system including a biological information acquisition device according to a first example embodiment. The biological information acquisition system 1 includes, as shown in FIG. 1, a camera 10 and a biological information acquisition device 100 connected to the camera 10 through a network 50 such as a WAN (Wide Area Network).

The camera 10 is provided, for example, in a device such as a personal computer, a smartphone, and a tablet type computer. Further, for example, when a predetermined application relating to measuring the SpO2 is activated, the camera 10 acquires a video (hereinafter, also referred to as a biological video) obtained by shooting a skin of an arbitrary position of a subject, and transmits the acquired video to the biological information acquisition device 100 via the network 50. Specifically, the camera 10 acquires a video obtained by shooting the surface of the hand or the face of the subject and transmits the acquired video to the biological information acquisition device 100, for example.

The biological information acquisition device 100 receives the biological video transmitted from the camera 10 via the network 50, and performs processing relating to estimation of the SpO2 of the subject based on the biological video. Also, the biological information acquisition device 100 accumulates the biological video received via the network 50 and the estimation results of the SpO2 of the subject. According to the present example embodiment, the estimation result of the SpO2 of the subject may be transmitted from the biological information acquisition device 100 to the device in which the camera 10 is provided through the network 50 to be notified to the subject. Further, according to the present example embodiment, the camera 10 and the biological information acquisition device 100 may be directly connected without passing through the network 50. Further, according to the present example embodiment, the processing relating to the estimation of the SpO2 of the subject may be performed in the device in which the camera 10 and the biological information acquisition device 100 are integrated.

### [Hardware Configuration]

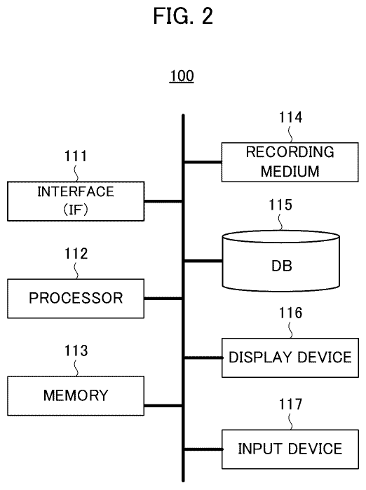

FIG. 2 is a block diagram illustrating a hardware configuration of the biological information acquisition device according to the first example embodiment. As shown in FIG. 2, the biological information acquisition device 100 includes an interface (IF) 111, a processor 112, a memory 113, a recording medium 114, a data base (DB) 115, a display device 116, and an input device 117.

The IF 111 inputs and outputs data to and from external devices. For example, the biological video transmitted from the camera 10 to the biological information acquisition device 100 through the network 500 is received by the IF 111.

The processor 112 is a computer such as a CPU (Central Processing Unit), and controls the whole of the biological information acquisition device 100 by executing a preliminarily prepared program. Specifically, the processor 112 performs processing relating to estimation of the SpO2 of the subject.

The memory 113 may include a ROM (Read Only Memory), a RAM (Random Access Memory), and the like. The memory 113 is also used as a working memory during various operations by the processor 112.

The recording medium 114 is a non-volatile and non-transitory recording medium such as a disk-like recording medium or a semiconductor memory and is detachably configured with respect to the biological information acquisition device 100. The recording medium 114 records various programs executed by the processor 112. When the biological information acquisition device 100 executes various processes, the program recorded in the recording medium 114 is loaded into the memory 113 and executed by the processor 112.

The DB 115 stores, for example, the biological video received by the IF 111 and the processing results obtained by the processing of the processor 112.

The display device 116 has a liquid crystal display or the like, for example. In addition, the display device 116 displays information such as the estimation result of the SpO2 of the subject, as needed.

The input device 117 includes, for example, at least one of a keyboard, a mouse, and a touch panel. The input device 117 gives the processor 112 the instructions according to an operation of a user such as a doctor.

### [Functional Configuration]

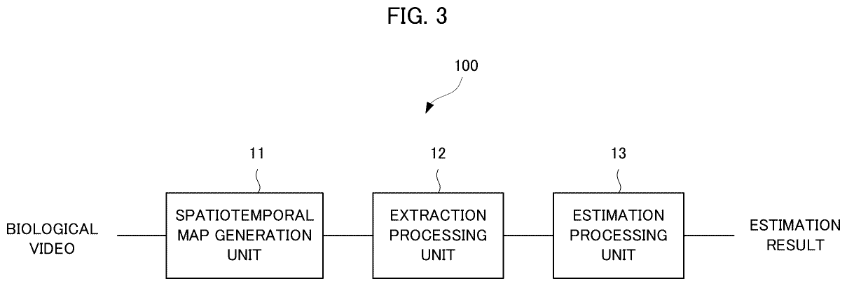

FIG. 3 is a block diagram showing a functional configuration of the biological information acquisition device according to the first example embodiment. As shown in FIG. 3, the biological information acquisition device 100 includes a spatiotemporal map generation unit 11, an extraction processing unit 12, and an estimation processing unit 13.

The spatiotemporal map generation unit 11 has a function as a spatiotemporal information generation means. The spatiotemporal map generation unit 11 extracts a plurality of regions of interest from a plurality of time-series images included in the biological video obtained by the camera 10, and generates a spatiotemporal map according to the intensity of each of red, green, and blue pixel included in the plurality of regions of interest.

The region of interest may be extracted as a region having a predetermined size and including, for example, a hand or a portion of a face of a subject. Further, it is preferable that the region of interest is extracted as a region having a predetermined resolution lower than the resolution of the biological video obtained by the camera 10, for example. Further, the region of interest may be extracted a predetermined number for each image included in the biological video obtained by the camera 10, for example.

The spatiotemporal map is generated, for example, as the accumulation of the intensity of each of red, green, and blue pixel included in the regions of interest extracted from each of the images in a predetermined period TA included in the biological video obtained by the camera 10. In other words, the spatiotemporal map includes information in the temporal direction corresponding to the acquisition frequency or the acquisition count of the time-series images included in the biological video obtained by the camera 10, and information in the spatial direction corresponding to the intensity of each of red, green, and blue pixel of the region of interest extracted from the images. Specifically, when extracting the region of interest from each of the 5-second images in 60 fps biological video, for example, the spatiotemporal map generation unit 11 generates a spatiotemporal map in which the data of the intensity of each of red, green, and blue pixel included in the region of interest is accumulated for 300 pieces in the temporal direction. In such a case, it is possible to acquire temporal information indicating that the intensity data is obtained from the n-th (1≤n≤300) image in the predetermined period TA. Further, when the resolution of the region of interest extracted from the n-th image is 16×14, for example, data of the intensity of 244 pixels for each of red, green and blue included in the region of interest can be acquired as the information in the spatial direction.

That is, the spatiotemporal map generation unit 11 generates a spatiotemporal map by accumulating information relating to the regions of interest extracted from a plurality of time-series images included in the biological video for a predetermined period TA.

The extraction processing unit 12 has a function as an extraction means. The extraction processing unit 12 extracts the AC component and the DC component corresponding to each of red, green, and blue from the spatiotemporal map generated by the spatiotemporal map generation unit 11 and outputs the extracted AC components and the DC components to the estimation processing unit 13.

The AC component is extracted as a frequency component derived from pulsatile arterial blood. In addition, the DC component is extracted as a frequency component derived from non-pulsatile arterial blood, venous blood, and other tissues.

The estimation processing unit 13 has a function as an estimation means. The estimation processing unit 13 includes, for example, a CNN (Convolutional Neural Network) model. The estimation processing unit 13 estimates the SpO2 of the subject in the predetermined period TA based on the AC components and the DC components extracted by the extraction processing unit 12. The estimation processing unit 13 outputs the information indicating the estimation result of the SpO2 of the subject in the predetermined period TA to at least one of the display device 116 and the device in which the camera 10 is provided.

### Specific Examples

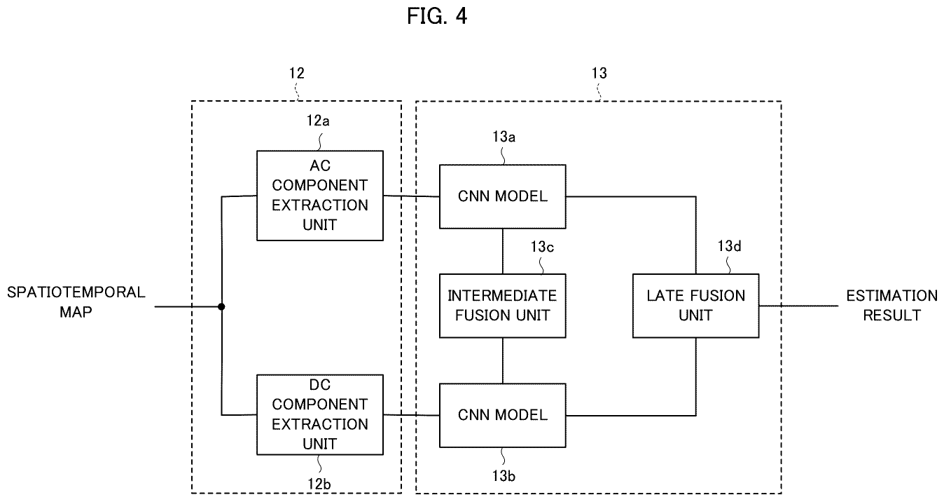

Subsequently, specific examples of the configuration of the extraction processing unit 12 and the estimation processing unit 13 will be described. FIG. 4 is a block diagram for explaining a specific configuration of the biological information acquisition device according to the first example embodiment. The extraction processing unit 12 includes an AC component extraction unit 12a and a DC component extraction unit 12b, as shown in FIG. 4. The estimation processing unit 13 includes a CNN model 13a, a CNN model 13b, an intermediate fusion unit 13c, and a late fusion unit 13d, as shown in FIG. 4. According to the configuration of the estimation processing unit 13, since the CNN models 13a and 13b for extracting the features of the AC and DC components can be designed, respectively, the SpO2 can be estimated with high accuracy compared with the configuration in which the data obtained by fusing the AC and DC components outputted from the extraction processing unit 12 is inputted into one CNN model, for example.

### Specific Example 1

The AC component extraction unit 12a extracts the AC component corresponding to each of red, green, and blue by applying a predetermined band-pass filter to the spatiotemporal map generated by the spatiotemporal map generation unit 11. Specifically, the AC component extraction unit 12a extracts the AC component corresponding to each of red, green, and blue by applying a band-pass filter to the time-series signal x∈R^{T} of the i-channel k-th region of interest, which is included in the spatiotemporal map X∈R^{3×K×T} (R: real number, 3: number indicating three channels of red, green, and blue, K: number of regions of interest, T: number of frame images). In other words, the AC component extraction unit 12a extracts the AC component corresponding to each of red, green, and blue by performing a filtering process of the spatiotemporal map generated by the spatiotemporal map generation unit 11. It is preferable that the predetermined band-pass filter has a characteristic to pass the frequency range larger than 0.75 Hz and smaller than 2.5 Hz and block the frequency range other than the above frequency range, for example. In addition, the AC component extraction unit 12a outputs the red AC component, the green AC component, and the blue AC component extracted as described above to the CNN model 13a. The AC component extraction unit 12a may extract the AC component corresponding to each of red, green, and blue by applying a filter other than a band-pass filter to the spatiotemporal map. In addition, the AC component extraction unit 12a may output the red AC component and the blue AC component extracted from the spatiotemporal map to the CNN model 13a.

The DC component extraction unit 12b extracts the DC component corresponding to each of red, green, and blue by applying a predetermined low-pass filter to the spatiotemporal map generated by the spatiotemporal map generation unit 11. Specifically, the DC component extraction unit 12B extracts the DC component corresponding to each of red, green, and blue by applying a low-pass filter to the time-series signal x∈R^{T} of the i-channel kth region of interest included in the spatiotemporal map x∈R^{3×K×T}. In other words, the DC component extraction unit 12b extracts the DC component corresponding to each of red, green, and blue by performing a filtering process of the spatiotemporal map generated by the spatiotemporal map generation unit 11. It is preferable that the predetermined low-pass filter has a characteristic to pass the frequency range below 0.3 Hz and block the frequency range equal to or higher than 0.3 Hz, for example. The DC component extraction unit 12b outputs the red DC component, the green DC component, and the blue DC component extracted as described above to the CNN model 13b. The DC component extraction unit 12b may extract the DC component corresponding to each of red, green, and blue by applying a filter other than the low-pass filter to the spatiotemporal map. In addition, the DC component extraction unit 12b may output the red DC component and the blue DC component extracted from the spatiotemporal map to the CNN model 13b.

The CNN model 13a is configured, for example, as a model with “ResNet18”. “ResNet18” is disclosed in Kaiming He, et. al, “Deep Residual Learning for Image Recognition,” in Proc. IEEE/CVF conf. Computer Vision and Pattern Recognition (CVPR) and 2016, pp. 770-778. The CNN model 13a calculates numerical data corresponding to the red AC component, the green AC component, and the blue AC component obtained by the AC component extraction unit 12a, and outputs the calculated numerical data to the late fusion unit 13d. The intermediate layer of the CNN model 13a is connected to the intermediate layer of the CNN model 13b through the intermediate fusion unit 13c. The CNN model 13a may be configured as a model different from “ResNet18.” In addition, the CNN model 13a may be configured as a pre-trained model for which images acquired from a large-scale data base such as “ImageNet” are used for training. “ImageNet” is disclosed in Jia Deng, et. al, “ImageNet: A large-Scale Hierarchical Image Database,” in Proc. IEEE/CVF conf. Computer Vision and Pattern Recognition (CVPR) and 2009, pp. 248-255.

The CNN model 13b is configured, for example, as a model with “ResNet18”. The CNN model 13b calculates numerical data corresponding to the red DC component, the green DC component, and the blue DC component obtained by the DC component extraction unit 12b, and outputs the calculated numerical data to the late fusion unit 13d. The intermediate layer of the CNN model 13b is connected to the intermediate layer of the CNN model 13a through the intermediate fusion unit 13c. The CNN model 13b may be configured as a model different from “ResNet18.” The CNN model 13b may be configured as a pre-trained model for which images acquired from a large-scale data base such as ImageNet are used for training.

The intermediate fusion unit 13c has “Multi-Modal Transfer Module (MMTM)”, for example. “MMTM” is disclosed in Hamid Reza Vaezi Joze, et. al, “MMTM: Multimodal Transfer Module for CNN Fusion,” in Proc. IEEE/CVF Conference on Computer Vision and Pattern Recognition (CVPR) and 2020, pp. 13289-13299. The intermediate fusion unit 13c acquires information on the parameters such as weights applied to the red AC component, the green AC components, and the blue AC components in the intermediate layer of the CNN model 13a and outputs the acquired information to the intermediate layer of the CNN model 13b. Also, the intermediate fusion unit 13c acquires information on the parameters such as weights applied to the red DC component, the green DC component, and the blue DC component in the intermediate layer of the CNN model 13b and outputs the acquired information to the intermediate layer of the CNN model 13a. When the CNN models 13a and 13b are configured as the models having “ResNet18”, for example, the intermediate fusion unit 13c may be configured such that information can be exchanged between the final layer in four blocks included in the intermediate layer of the CNN model 13a and the final layer in four blocks included in the intermediate layer of the CNN model 13b. The intermediate fusion unit 13c may be configured to exchange information between the intermediate layer of the CNN model 13a and the intermediate layer of the CNN model 13b by a method different from MMTM.

The late fusion unit 13d estimates the SpO2 of the subject in the predetermined term TA based on the data in which the numerical data obtained by the CNN model 13a and the numerical data obtained by the CNN model 13b were fused. Specifically, the late fusion unit 13d estimates the SpO2 of the subject in the predetermined period TA based on 2×P numerical data obtained by fusing P numerical data obtained by the CNN model 13a and P numerical data obtained by the CNN model 13b, for example. More specifically, when the predetermined time TA is set to 10 seconds, for example, the later fusing unit 13d converts the 2×P numerical data into 10 data through the linear layer, and outputs the 10 data as the estimation result of the SpO2 of the subject in 10 seconds. In such a case, the linear layer is trained by the training method described later.

Here, according to this specific example, in order to estimate the SpO2 of the subject in the predetermined period TA, each part belonging to the estimation processing unit 13 is trained in a predetermined manner. Specifically, according to this specific example, the training using the loss-function Lₛₚ representing the estimation error of the SpO2 is performed for each part belonging to the estimation processing unit 13, as shown in Equation (1) below. In Equation (1) below, yₒᵤₜ∈R^{S} represents the output value of the estimation processing unit 13 (the late fusion unit 13d), and y_{GT}∈R^{S} represents the correct value of the SpO2 corresponding to the output value (S: the output number of the SpO2 in the predetermined period TA), for example. It is preferable that yₒᵤₜ and y_{GT} are values normalized in accordance with a predetermined rule. In addition, the first term on the right-hand side of Equation (1) below represents the mean squared error of yₒᵤₜ and y_{GT} multiplied by the hyperparameter α. The second term on the right-hand side of Equation (1) represents the negative Pearson's coefficient of correlation in yₒᵤₜ and y_{GT} multiplied by the hyperparameter β.

$$[ Equation 1 ]$$

$${L}_{sp} = a \cdot MSE ( {y}_{out} , {y}_{GT} ) + β \cdot NegCorr ( {y}_{out} , {y}_{GT} ) \qquad ( 1 )$$

According to this specific example, while reducing the estimation error obtained by the first term on the right side of Equation (1), each part of the estimation processing unit 13 is trained so as to increase the degree of similarity of SpO2 obtained by the second term on the right side of Equation (1). According to this training, the CNN models 13a and 13b can extract features useful for estimating the subject's SpO2 by exchanging information relating to the red, green, and blue DC components and the red, green, and blue AC components via the intermediate fusion 13c. Further, according to the above-described training, the late fusion unit 13d can estimate the SpO2 of the subject in a predetermined period TA by using the numerical data outputted from the CNN models 13a and 13b.

### Specific Example 2

In the above-described specific example 1, the extraction processing unit 12 extracts the AC components and the DC components by performing the filtering processing on the spatiotemporal map generated by the spatiotemporal map generation unit 11. Instead, in the second specific example, the extraction processing unit 12 extracts the AC components and the DC components from the spatiotemporal map using a deep learning model.

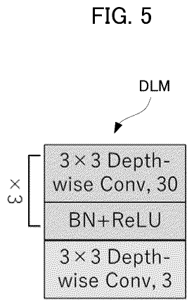

Specifically, the AC component extraction unit 12a extracts the AC component corresponding to each of red, green, and blue by inputting the spatiotemporal map generated by the spatiotemporal map generation unit 11 into the deep learning model DLM having the configuration as shown in FIG. 5. In addition, the AC component extraction unit 12a outputs the red AC component, the green AC component, and the blue AC component extracted as described above to the CNN model 13a.

The DC component extraction unit 12b extracts the DC component corresponding to each of red, green, and blue by inputting the spatiotemporal map generated by the spatiotemporal map generation unit 11 into the deep learning model DLM having the configuration as shown in FIG. 5. In addition, the DC component extraction unit 12b outputs the red DC component, the green DC component, and the blue DC component extracted as described above to the CNN model 13b.

FIG. 5 is a diagram showing a specific example of a deep learning model used for extracting the AC component and the DC component by the biological information acquisition device according to the first example embodiment. The deep learning model DLM is provided separately in the AC component extraction unit 12a and the DC component extraction unit 12b. The “Depth-wise Conv” of the deep learning model DLM corresponds to the abbreviation of Depth-wise Convolution. The “BN” of the deep learning model DLM corresponds to the abbreviation of Batch Normalization. The “ReLU” of the deep learning model DLM corresponds to the abbreviation of Rectified Linear Unit. Further, the deep learning model DLM is configured to perform processing of “3×3 Depth-wise Conv,3” after performing processing of “3×3 Depth-wise Cony, 30” and “BN+ReLU” three times repeatedly according to the input of the spatiotemporal map. Note that the “3×3” described at the beginning of the above-mentioned processing in the double quotation marks represents the kernel size of the convolution layer. Further, “30” and “3” described at the end of the processing in the double quotation marks represent the number of channels of the convolution layer. According to the deep learning model DLM, convolution can be performed for each of the red, green, and blue channels of the spatiotemporal map generated by the spatiotemporal map generation unit 11. Therefore, the AC component extraction unit 12a can acquire the processing result of the convolution performed in the deep learning model DLM as the red AC component, the green AC component, and the blue AC component. In addition, the DC component extraction unit 12b can acquire the processing result of the convolution performed in the deep learning model DLM as the red DC component, the green DC component, and the blue DC component. According to this specific example, the AC components and the DC components may be extracted using another deep learning model having a configuration different from the deep learning model DLM.

Namely, the AC component extraction unit 12a of this specific example extracts the AC components corresponding to each of red, green, and blue by inputting the spatiotemporal map into the deep learning model DLM and performing depth-wise convolution for each of the red, green, and blue channels. Also, the DC component extraction unit 12b of this specific example extracts the DC components corresponding to each of red, green, and blue by inputting the spatiotemporal map into the deep learning model DLM and performing depth-wise convolution for each of the red, green, and blue channels.

The estimation processing unit 13 of this specific example has the same configuration as that described in the specific example 1. Therefore, in this specific example, description of the specific configuration of the estimation processing unit 13 will be omitted.

Here, according to this specific example, in order to estimate the SpO2 of the subject in a predetermined period TA, each part belonging to the extracting processing unit 12 and the estimating processing unit 13 is trained in a predetermined manner. Specifically, according to this specific example, the training using the loss function L_{E} shown in the following Equation (2) is performed for each part belonging to the extraction processing unit 12 and the estimation processing unit 13. In the following Equation (2), X_{ACC} represents the output value of the AC component extraction unit 12a, X_{ACF} represents the correct value of the AC component extracted by applying the filter to the spatiotemporal map, X_{DCC} represents the output value of the DC component extraction unit 12b, and X_{DCF} represents the correct value of the DC component extracted by applying the filter to the spatiotemporal map. The first term on the right side of Equation (2) is the same as Lₛₚ of Equation (1). The second term on the right-hand side of Equation (2) represents the mean squared error of X_{ACC} and X_{ACF} multiplied by the hyperparameter γ. The third term on the right-hand side of Equation (2) represents the mean squared error of X_{DCC} and X_{DCF} multiplied by the hyperparameter γ.

$$[ Equation 2 ]$$

$${L}_{E} = {L}_{sp} + γ \cdot MSE ( {X}_{ACC} , {X}_{ACF} ) + γ \cdot MSE ( {X}_{DCC} , {X}_{DCF} ) \qquad ( 2 )$$

According to this specific example, each part belonging to the extraction processing unit 12 and the estimation processing unit 13 is trained so that the estimation error of the SpO2 obtained by the first term on the right-hand side of Equation (2), the estimation error of the AC component obtained by the second term on the right-hand side of Equation (2), and the estimation error of the DC component obtained by the third term on the right-hand side of Equation (2) are adjusted simultaneously. That is, the extraction processing unit 12 and the estimation processing unit 13 of this specific example are trained by using a loss function including an estimation error of the blood oxygen saturation, an estimation error of the AC component, and an estimation error of the DC component. Further, according to this specific example, it is possible to perform training while adjusting the weights of the estimation error of the SpO2, and the estimation errors of the AC components and the DC components extracted from the spatiotemporal map, by the hyperparameter γ. According to such training, the AC component extraction unit 12a can extract the AC components including the features which are considered to be useful for estimating the SpO2 from the spatiotemporal map. Further, according to the above-described training, the DC component extraction unit 12b can extract the DC components including the features which are considered to be useful for estimating the SpO2 from the spatiotemporal map. Further, according to the above-described training, the SpO2 of the subject in a predetermined period TA can be estimated with high accuracy by processing of each part belonging to the estimation processing unit 13.

### [Processing Flow]

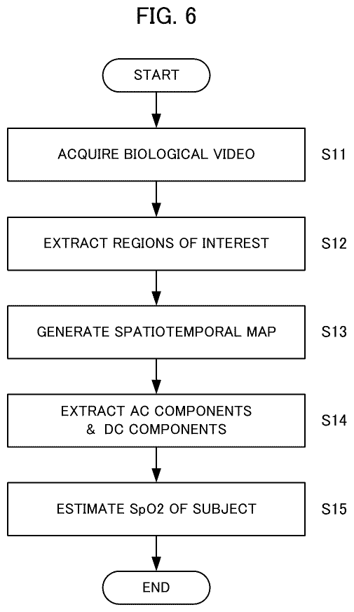

Next, a flow of the processing performed in the biological information acquisition device according to the first example embodiment will be described. FIG. 6 is a flowchart illustrating an example of processing performed in the biological information acquisition device according to the first example embodiment.

First, the biological information acquisition device 100 acquires a biological video transmitted from the camera 10 to the biological information acquisition device 100 through the network 500 (step S11)

Next, the biological information acquisition device 100 extracts a plurality of regions of interest from a plurality of time-series images included in the biological video obtained in step S11 (step S12).

Subsequently, the biological information acquisition device 100 generates a spatiotemporal map using the plurality of regions of interest extracted in step S12 (step S13).

Subsequently, the biological information acquisition device 100 extracts the AC component and the DC component corresponding to each of red, green and blue from the spatiotemporal map generated in step S13 (step S14).

Subsequently, the biological information acquisition device 100 estimates the SpO2 of the subject based on the AC components and the DC components extracted in step S14 (step S15).

As described above, according to the present example embodiment, it is possible to extract the AC components and the DC components from the spatiotemporal map including the information in the temporal direction and the information in the spatial direction, and estimate the SpO2 of the subject based on the extracted AC components and DC components. Therefore, according to the present example embodiment, it is possible to improve the accuracy of estimating the blood oxygen saturation.

Further, according to the present example embodiment, it is possible to estimate the SpO2 of the subject without contact, i.e., without using a contact-type device such as a pulse oximeter or the like.

### [Modifications]

Hereinafter, modifications to the above example embodiment will be described.

According to the present example embodiment, the intermediate fusion unit 13c may not be provided in the estimation processing unit 13.

Further, according to the present example embodiment, the AC components and the DC components outputted from the extraction processing unit 12 may be inputted to one CNN model in the estimation processing unit 13.

Further, according to the present example embodiment, the spatiotemporal map generation unit 11 may generate the spatiotemporal map using a plurality of biological videos obtained by a plurality of cameras 10.

### Second Example Embodiment

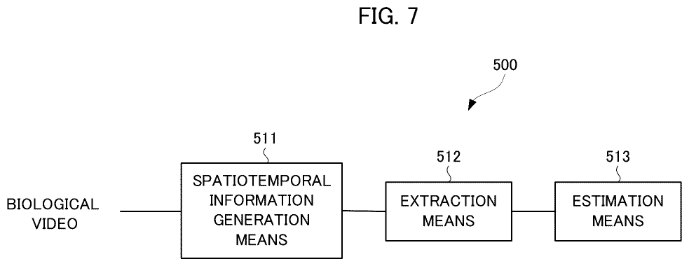

FIG. 7 is a block diagram showing a functional configuration of a biological information acquisition device according to a second example embodiment.

The biological information acquisition device 500 according to this example embodiment has the same hardware configuration as the biological information acquisition device 100. Further, the biological information acquisition device 500 includes a spatiotemporal information generation means 511, an extraction means 512, and an estimation means 513.

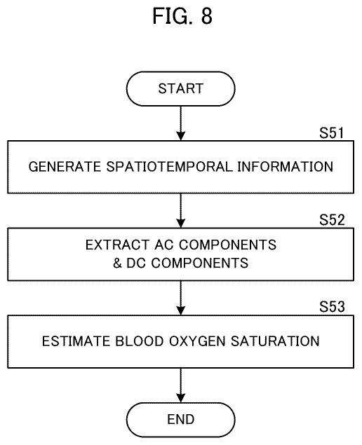

FIG. 8 is a flow chart for describing processing performed in the biological information acquisition device according to the second example embodiment.

The spatiotemporal information generation means 511 generates spatiotemporal information by accumulating information relating to a region of interest extracted from a plurality of time-series images included in a biological video, the biological video being obtained by shooting a skin of a subject for a predetermined period (step S51).

The extraction means 512 extracts AC components and DC components from the spatiotemporal information (step S52).

The estimation means 513 estimates blood oxygen saturation of the subject in the predetermined period, based on the AC components and the DC components (step S53).

According to this example embodiment, it is possible to improve the estimation accuracy of the blood oxygen saturation.

A part or all of the example embodiments described above may also be described as the following supplementary notes, but not limited thereto.

### (Supplementary Note 1)

A biological information acquisition device comprising:
a spatiotemporal information generation means configured to generate spatiotemporal information by accumulating information relating to a region of interest extracted from a plurality of time-series images included in a biological video, the biological video being obtained by shooting a skin of a subject for a predetermined period;
an extraction means configured to extract AC components and DC components from the spatiotemporal information; and
an estimation means configured to estimate blood oxygen saturation of the subject in the predetermined period, based on the AC components and the DC components.

### (Supplementary Note 2)

The biological information acquisition device according to Supplementary note 1, wherein the extraction means extracts the AC components and the DC components by applying filtering processing on the spatiotemporal information.

### (Supplementary Note 3)

The biological information acquisition device according to Supplementary note 1, wherein the extraction means extracts the AC components and the DC components by inputting the spatiotemporal information into a deep learning model.

### (Supplementary Note 4)

The biological information acquisition device according to Supplementary note 3, wherein the extraction means and the estimation means are trained by using a loss function including an estimation error of the blood oxygen saturation, an estimation error of the AC components, and an estimation error of the DC components.

### (Supplementary Note 5)

The biological information acquisition device according to Supplementary note 3,
wherein the spatiotemporal information includes information in a temporal direction corresponding to an acquisition frequency or an acquisition count of the time-series images included in the biological video, and information in a spatial direction corresponding to intensity of each of red, green and blue pixels of the region of interest, and
wherein the extraction means extracts the AC components and the DC component by inputting the spatiotemporal information into the deep learning model to perform convolution for each of red, green, and blue channels.

### (Supplementary Note 6)

The biological information acquisition device according to Supplementary note 5, wherein the convolution is depth-wise convolution.

### (Supplementary Note 7)

The biological information acquisition device according to Supplementary note 1, wherein the estimation means is trained using a loss function representing an estimation error of the blood oxygen saturation.

### (Supplementary Note 8)

The biological information acquisition device according to Supplementary note 1, wherein the biological video is a video obtained by shooting a surface of a hand or a face of the subject.

### (Supplementary Note 9)

A biological information acquisition method comprising:
generating spatiotemporal information by accumulating information relating to a region of interest extracted from a plurality of time-series images included in a biological video, the biological video being obtained by shooting a skin of a subject for a predetermined period;
extracting AC components and DC components from the spatiotemporal information; and
estimating blood oxygen saturation of the subject in the predetermined period, based on the AC components and the DC components.

### (Supplementary Note 10)

A recording medium storing a program, the program causing a computer to execute processing comprising:
generating spatiotemporal information by accumulating information relating to a region of interest extracted from a plurality of time-series images included in a biological video, the biological video being obtained by shooting a skin of a subject for a predetermined period;
extracting AC components and DC components from the spatiotemporal information; and
estimating blood oxygen saturation of the subject in the predetermined period, based on the AC components and the DC components.

While the present disclosure has been described with reference to the example embodiments, the present disclosure is not limited to the above example embodiments and examples. Various changes which can be understood by those skilled in the art within the scope of the present disclosure can be made in the configuration and details of the present disclosure.

### DESCRIPTION OF SYMBOLS

11 Spatiotemporal map generation unit
12 Extraction processing unit
13 Estimation processing unit
100 Biological information acquisition system

What is claimed is:

1. A biological information acquisition device comprising:
a memory configured to store instructions; and
a processor configured to execute the instructions to:
generate spatiotemporal information by accumulating information relating to a region of interest extracted from a plurality of time-series images included in a biological video, the biological video being obtained by shooting a skin of a subject for a predetermined period;
extract AC components and DC components from the spatiotemporal information; and
estimate blood oxygen saturation of the subject in the predetermined period, based on the AC components and the DC components.

2. The biological information acquisition device according to claim 1, wherein the processor extracts the AC components and the DC components by applying filtering processing on the spatiotemporal information.

3. The biological information acquisition device according to claim 1, wherein the processor extracts the AC components and the DC components by inputting the spatiotemporal information into a deep learning model.

4. The biological information acquisition device according to claim 3, wherein the processor is trained by using a loss function including an estimation error of the blood oxygen saturation, an estimation error of the AC components, and an estimation error of the DC components.

5. The biological information acquisition device according to claim 3,
wherein the spatiotemporal information includes information in a temporal direction corresponding to an acquisition frequency or an acquisition count of the time-series images included in the biological video, and information in a spatial direction corresponding to intensity of each of red, green and blue pixels of the region of interest, and
wherein the processor extracts the AC components and the DC component by inputting the spatiotemporal information into the deep learning model to perform convolution for each of red, green, and blue channels.

6. The biological information acquisition device according to claim 5, wherein the convolution is depth-wise convolution.

7. The biological information acquisition device according to claim 1, wherein the processor is trained using a loss function representing an estimation error of the blood oxygen saturation.

8. The biological information acquisition device according to claim 1, wherein the biological video is a video obtained by shooting a surface of a hand or a face of the subject.

9. A biological information acquisition method comprising:
generating spatiotemporal information by accumulating information relating to a region of interest extracted from a plurality of time-series images included in a biological video, the biological video being obtained by shooting a skin of a subject for a predetermined period;
extracting AC components and DC components from the spatiotemporal information; and
estimating blood oxygen saturation of the subject in the predetermined period, based on the AC components and the DC components.

10. A non-transitory computer-readable recording medium storing a program, the program causing a computer to execute processing comprising:
generating spatiotemporal information by accumulating information relating to a region of interest extracted from a plurality of time-series images included in a biological video, the biological video being obtained by shooting a skin of a subject for a predetermined period;
extracting AC components and DC components from the spatiotemporal information; and
estimating blood oxygen saturation of the subject in the predetermined period, based on the AC components and the DC components.
